(19) United States
(12) Patent Application Publication
(10) Pub. No.: US 2025/0341518 A1
(43) Pub. Date: Nov. 6, 2025

(54) MAGNETIC MICROGEL BEADS, METHODS OF MAKING AND USES THEREOF

(71) Applicant: MCMASTER UNIVERSITY, Hamilton, Ontario (CA)

(72) Inventors: Leyla SOLEYMANI, Oakville, Ontario (CA); Todd HOARE, Ancaster, Ontario (CA); Yingfu LI, Dundas, Ontario (CA); Yang LU, Linxia City, Gansu (CN); Richa PANDEY, Hamilton, Ontario (CA)

(21) Appl. No.: 18/710,416

(22) PCT Filed: Nov. 14, 2022
(86) PCT No.: PCT/CA2022/051679

§ 371 Date: May 15, 2024

Related U.S. Application Data
(60) Provisional application No. 63/279,417, filed on Nov. 15, 2021.

(51) Int. Cl. G01N 33/543; G01N 33/553

(52) U.S. Cl. CPC G01N 33/5434; G01N 33/553

(57) ABSTRACT

This disclosure relates to magnetic microgel beads, and in particular to magnetic microgel beads for biofunctionalization and methods of making and uses thereof, for example, in biosensing assays. In an embodiment, a magnetic microparticle comprising a magnetic nanoparticle encapsulated by a polymer hydrogel. In another embodiment, an assay for detecting the presence of a target analyte in a sample comprising a) the magnetic microparticle disclosed herein, wherein the biorecognition agent further comprises a reporter moiety; b) an electrochemical chip comprising a working electrode, a counter electrode and a reference electrode; and c) a capture probe functionalized on the working electrode; wherein binding of the biorecognition agent to the target analyte results in production of an electrochemical, electroluminescent or photoelectrochemical signal.

### CROSS-REFERENCE TO RELATED APPLICATIONS

This application is the 35 U.S.C. § 371 national stage application of PCT Application No. PCT/CA2022/051679, filed Nov. 14, 2022, where the PCT claims priority to, and the benefit of, U.S. Provisional Application No. 63/279,417, filed Nov. 15, 2021, both of which are herein incorporated by reference in their entireties.

### REFERENCE TO SEQUENCE LISTING

The Sequence Listing submitted May 15, 2024, as an Extensible Markup Language file named “2025-01-08_Sequence_Listing_330106-1000_Filed.xml,” created on Jan. 8, 2025, and having a size of 9,890 bytes is hereby incorporated by reference.

### FIELD

The present disclosure relates to magnetic microgel beads, and in particular to magnetic microgel beads for biofunctionalization and methods of making and uses thereof, for example, in biosensing assays.

### BACKGROUND

There is an urgent need for rapid and facile infectious disease tests that can be operated at the point-of-care (POC) for expediting and improving clinical decision making. Electrochemical biosensors enable sensitive signal readout using inexpensive and handheld instrumentation, thus making these systems ideally suited for POC diagnostics. However, in spite of the abundance of reports demonstrating the sensitive and specific electrochemical detection of processed bimolecular targets of infectious diseases, i.e. proteins and nucleic acids, the direct and rapid analysis of clinical samples without enrichment, purification, and/or the addition of reagents remains elusive. Conventional electrochemical biosensors employ electrodes as the sole site for target analyte capture and signal transduction. This approach has two key drawbacks: (1) target analytes must diffuse through the bulk of the solution to reach the biorecognition elements immobilized on the heterogeneous electrode surface, limiting the probability of probe/target interaction and (2) typical strategies used to reduce the non-specific adsorption of fouling chemicals on surfaces also significantly reduce charge transfer and thus the resultant signal transduction efficiency of the electrodes, resulting in either inherently lower sensitivity or high biofouling that over time reduces both sensitivity and selectivity. Some electrode surface coatings have also been reported for the high-sensitivity detection of bacterial nucleic acid biomarkers but require pre-processed bacterial samples (including bacterial lysates) and are mostly limited to use on gold electrodes given the frequent use of thiol-based surface functionalization chemistry.

To overcome the drawbacks of electrode-based capture and signal transduction, microbeads functionalized with a capture ligand may be used to allow target capture to occur away from the electrode while signal transduction occurs on the electrode surface. The vast majority of bead-based biological assays employ commercially-available magnetic beads with polymeric shells covering a magnetic core to enable sensing of the contents of a sample solution without potential interference from the microbead. However, these magnetic beads typically have “hard” silica or polystyrene shells that result in poorly hydrated interfaces, introducing steric challenges associated with target binding and biofouling with unwanted background materials. While post-synthesis modification methods such as functionalization with glycidyl ether have been used to increase the hydrophilicity of these commercial beads, their non-porous surface limits the number of binding sites available per bead.

In contrast, microgel beads comprised of crosslinked water-soluble polymers offer controllable porosity (based on the crosslinker concentration used) and easily tunable functionality while maintaining a highly hydrated interface. Collectively, these properties reduce mass transport barriers between the bead and the solution to promote higher ligand conjugation efficiencies, solution state-like ligand conformations, and easier access of targets to binding sites throughout the microgel, all beneficial for promoting higher binding sensitivity and selectivity. While magnetic microgels have been investigated in the areas of drug delivery (particularly cancer therapy), gene delivery, bioseparations, biocatalysis, and regenerative medicine, to-date demonstrations of any type of magnetic microgel in an integrated biosensing platform remain elusive. In particular, the small sizes (<250 nm) and/or low degrees of magnetization of most reported magnetic microgels make them challenging to apply in a point-of-care biosensor application in which the use of strong electromagnets and/or longer-than-acceptable separation times for magnetic separation is not practically feasible.

The background herein is included solely to explain the context of the disclosure. This is not to be taken as an admission that any of the material referred to was published, known, or part of the common general knowledge as of the priority date.

### SUMMARY

The present disclosure describes, in aspects, microgel magnetic beads for immobilizing biomolecules, such as DNAzyme programmed for electrochemical signal transduction, into a hydrated and three-dimensional scaffold, integrated in a target detection assay platform with electrodes for electrochemical readout to achieve rapid biosensing.

In accordance with an aspect, there is a magnetic microparticle comprising a magnetic nanoparticle encapsulated by a polymer hydrogel.

In aspects, the hydrogel comprises a three dimensional crosslinked network of water-soluble polymer(s).

In aspects, the polymer hydrogel comprises a protein repellent polymer.

In aspects, the hydrogel polymer comprises poly(oligo(ethylene glycol) methacrylate or a poly(ethylene glycol) derivative.

In aspects, the hydrogel polymer comprises a zwitterionic polymer; optionally, the zwitterionic polymer is selected from the group consisting of polysulfobetaine(s), poly(sulfobetaine) methacrylate, polycarboxybetaine(s), poly(carboxybetaine) methacrylate, and poly(phosporylcholine).

In aspects, the hydrogel polymer comprises poly(N-vinylpyrrolidone), poly(acrylamide) and poly(acrylamide) derivatives, polyglycidol and polyglycidol derivatives, or poly(2-oxazoline) or poly(2-oxazoline) derivatives.

In aspects, the microparticle is a microgel.

In aspects, the microgel comprises at least one dimension on the length scale of about 10 nm to about 1000 μm.

In aspects, the at least one dimension on the length scale is at least about 5 μm.

In aspects, the magnetic nanoparticle comprises iron oxide.

In aspects the microparticle is from about 0.5 μm to about 100 μm in diameter.

In aspects, the microparticle is at least about 5 μm in diameter.

In aspects, the microparticle is prepared by inverse emulsion templating.

In aspects, the magnetic microparticle further comprises a biorecognition agent functionalized on and/or in the microparticle.

In aspects, the biorecognition agent is at least one of a DNAzyme, an aptamer, and an antibody.

In accordance with another aspect, there is an assay for detecting the presence of a target analyte in a sample comprising:
a) the magnetic microparticle described herein, wherein the biorecognition agent further comprises a reporter moiety;
b) an electrochemical chip comprising a working electrode, a counter electrode and a reference electrode; and
c) a capture probe functionalized on the working electrode;
wherein binding of the biorecognition agent to the target analyte results in production of an electrochemical, electroluminescent or photoelectrochemical signal.

In aspects, the electrochemical signal is measured by amperometry, voltammetry, photoelectrochemistry, electrochemiluminescence, potentiometry or impedance.

In aspects, the working electrode comprises a conductive material, semi-conductive material, or a combination thereof.

In aspects, the working electrode comprises metal.

In aspects, the working electrode comprises gold.

In aspects, the working electrode further comprises hierarchical structures.

In aspects, the biorecognition agent is at least one of a DNAzyme, an aptamer, and an antibody.

In aspects, the reporter moiety comprises at least one of a redox species, a photoactive species, and a electrochemiluminescence species.

In aspects, the redox species is methylene blue.

In aspects, the reporter moiety comprises a biopolymer modified with the redox species.

In aspects, the biopolymer comprises single-stranded DNA.

In aspects, the capture probe comprises single-stranded DNA.

In aspects, the target analyte comprises a microorganism target.

In aspects, the microorganism is Escherichia coli.

In aspects, the sample is a urine sample.

In aspects, the urine sample is an unprocessed urine sample.

In aspects, the target analyte is detected in the sample in an amount of about 10 CFU/mL to about 10⁶ CFU/mL.

In aspects, the assay has a limits-of-detection for the target analyte of from about 50 CFU/mL to about 200 CFU/mL.

In aspects, the assay has a limits-of-detection for the target analyte of about 138 CFU/mL.

In aspects, the assay is performed within about 30 minutes to about 10 hours; about 30 minutes to about 8 hours; about 30 minutes to about 7 hours; about 30 minutes to about 6 hours; about 30 minutes to about 5 hours; about 30 minutes to about 4 hours; about 30 minutes to about 3 hours; about 30 minutes to about 2 hours; about 30 minutes to about 1 hour; about 45 minutes to about 1 hour; or about 1 hour.

In aspects, the assay is performed within about 1 hour.

In aspects, the assay is for use in screening and/or diagnostics, treatment monitoring, environmental monitoring, health monitoring, and/or pharmaceutical development.

In aspects, the assay detects a urinary tract infection in a subject.

In another aspect, there is a kit for detecting the presence of a target analyte in a sample, wherein the kit comprises
d) the magnetic microparticle described herein, wherein the biorecognition agent further comprises a reporter moiety;
e) an electrochemical chip comprising a working electrode, a counter electrode and a reference electrode;
f) a capture probe functionalized on the working electrode;
g) a magnet; and
h) instructions for use of the kit.

In aspects, the kit comprises a sample container and an electrical reader.

In yet another aspect, there is a method of determining the presence of a target analyte in a sample, comprising:
i) exposing the magnetic microparticle of the assay described herein to the sample to release the reporter moiety from the biorecognition agent in the presence of the target analyte;
j) separating the magnetic microparticle from the sample; and
k) depositing the sample of step b) to the electrochemical chip of the assay described herein;
wherein the capture probe of the assay described herein binds the reporter moiety to produce an electrochemical signal.

In aspects, the electrochemical signal is measured by square wave voltammetry.

In aspects, the magnetic microparticle is exposed to the sample under conditions for binding the biorecognition agent to the target analyte.

In aspects, the biorecognition agent is at least one of a DNAzyme, an aptamer, and an antibody.

In aspects, the target analyte comprises a microorganism target.

In aspects, the microorganism is Escherichia coli.

In aspects, the sample is a urine sample.

In aspects, the method detects a urinary tract infection in a subject.

In yet another aspect, there is a use of the magnetic microparticle described herein to capture a target analyte in a sample.

In yet another aspect, there is a use of the magnetic microparticle described herein to determine the presence of a target analyte in a sample.

In yet still another aspect, there is a use of the assay described herein to determine the presence of a target analyte in a sample.

In yet still another aspect, there is a use of the kit described herein to determine the presence of a target analyte in a sample.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### DRAWINGS

Certain embodiments of the disclosure will now be described in greater detail with reference to the attached drawings in which:

FIG. 1A-B shows a schematic of (A) the assay process from sample collection to assay result readout and (B) the E. coli mMB assay building blocks, in exemplary embodiments of the disclosure: (a) RNA-cleaving DNAzymes (DNAzyme) interact with the specific bacterial targets, releasing the redox DNA barcode tagged with methylene blue; (b) microgel magnetic beads (mMBs) composed of poly-oligo(ethylene glycol methacrylate) (POEGMA) and superparamagnetic iron oxide nanoparticles (SPIONS); (c) electrochemical chip (e-Chip) with a hierarchically structured working electrode.

FIG. 2 shows the base-into-acid conductometric titration curve—the x axis represents the amount of sodium hydroxide added while the y axis represents the conductivity of mixture—in exemplary embodiments of the disclosure.

FIG. 3 shows physical characterization of microgel magnetic beads in exemplary embodiments of the disclosure: (a) laser diffraction particle size distribution of 56 mol % methacrylic acid functionalized mMB; (b) optical microscope image of 56 mol % methacrylic acid functionalized mMBs (20 μm scale bar); (c) picture of mMB suspension before magnetic separation (left) and after 5 minutes of magnetic separation using a neodymium magnet (right); (d) scanning electron micrograph of a 70 mol % methacrylic acid functionalized mMB (2 μm scale bar); (e) thermogravimetric analysis for 70 mol % methacrylic acid functionalized mMBs; (f, g) particle stability over a one-month period as tracked by changes in electrophoretic mobility (f) and particle size (g) for sample stored at room temperature (error bars represent the standard deviation, n=3 independent preparations).

FIG. 4 shows a comparison of grafted fluorescein-labelled DNAzyme grafting efficiency and activity using magnetic microgel beads (mMB) compared to commercial Dynabeads (cMB) in exemplary embodiments of the disclosure: (a) residual DNAzyme in solution following carbodiimide-mediated grafting to mMBs showing improved immobilization of DNAzyme on mMBs relative to cMBs; (b) concentration of released DNA barcodes following exposure to the E. coli bacterial target in the presence of different concentrations of the grafted DNAzyme, demonstrating improved DNAzyme activity upon grafting to mMBs versus cMBs—circles and squares represent individual data points, and the error bar represents the standard deviation (n=3 independently prepared samples).

FIG. 5 shows electrochemical characterization of the reproducibility of the working electrode, capture probe deposition, e-Chip, and DNAzyme-mMBs in exemplary embodiments of the disclosure: (a) cyclic voltametric cleaning of the working electrode in 0.1M H₂SO₄ (40 cycles); (b) post-cleaning (i), post-probe deposition (iii) and post-MCH deposition (ii) validation of the three individual working electrodes (n=3) using cyclic voltammetry in 2 mM potassium hexacyanoferrate (II) redox solution; (c) reproducibility of the detection of an E. coli load of 105 CFU/mL in PMT 20 buffer on three independently-fabricated e-Chips (n=3); (d) reproducibility of the detection of an E. coli load of 10⁵ CFU/mL in PMT 20 buffer using three independently-fabricated batches of DNAzyme-mMBs.

FIG. 6 shows E. coli quantification using microgel magnetic beads coupled with nanostructured electrodes and electrochemical readout in exemplary embodiments of the disclosure: (a) schematic of the E. coli mMB kit comprised of a 3D printed holder (I) with incubation and magnetic separation slots for a reaction tube containing DNAzyme-grafted mMBs; bacterial detection is performed using a two-step process: (II) in step 1, the target solution is added to the reaction cube containing DNAzyme-modified mMBs, then following a 30-minute incubation time, the reaction tube is moved from the incubation to the magnetic separation slot and (III) after magnetic separation, a drop of the solution is positioned on the e-Chip and incubated for 30 minutes for signal readout; in the presence of the specific bacterial target, redox DNA barcodes are released in the reaction tube via DNAzyme cleavage, transferred to the e-Chip, and hybridized with the single stranded DNA probe on the working electrode of the e-Chip to generate a strong electrochemical peak measured using square wave voltammetry; (b) peak current density measured using the E. coli mMB kit with mMBs (grey bars) and cMB (black bars) in the presence of buffer spiked with different E. coli concentrations; (c) peak current density measured using the E. coli mMB kit at an E. coli concentration of 10⁵ CFU/mL spiked in buffer (grey bars) or undiluted urine (yellow bars) using mMBs and cMBs (in (b) and (c), circles and squares represent individual data points, and the error bar represents the standard deviation; n=3 independently prepared samples).

FIG. 7 shows kinetics of the E. coli mMB assay (1000 CFU/mL E. coli spiked urine) in exemplary embodiments of the disclosure: (a) current density measured over different time intervals of Step 1 of the mMB assay involving E. coli target incubation with DNAzyme-functionalized mMBs (Step 2 of electrochemical detection was performed over 30 min for all Step 1 time intervals tested); (b) current density measured over different time intervals of Step 2 of the mMB assay involving deposition of the supernatant obtained from Step 1 on the e-Chip (Step 1 of bacteria incubation with DNAzyme-functionalized mMBs was performed for 30 min for all Step 2 time intervals tested)—circles represent individual data points, and the error bar represents the standard deviation (n=3 independently prepared samples).

FIG. 8 shows a comparison of the anti-fouling/protein repellency of mMBs and cMBs in exemplary embodiments of the disclosure: percentage of bovine serum albumin (BSA), immunoglobin G (IgG), fibrinogen, or lysozyme binding to mMBs compared to cMBs (8 μg protein added per 20 μg of magnetic beads)—the amount of protein remaining in the supernatant after magnetic washing was measured to calculate the percentage of bound protein to the magnetic bead (circles and squares represent individual data points, and the error bar represents the standard deviation; n=3 independently prepared samples).

FIG. 9 shows the analytical and specificity performance of the E. coli mMB kit in exemplary embodiments of the disclosure: (a, b) square wave voltammograms measured using the E. coli mMB kit at various concentrations of E. coli in buffer (a) or unprocessed urine (b); (c,d) calibration curves of E. coli detection in buffer ((c), y=8×10⁻⁵e1.5×, R²=0.97) and unprocessed urine ((d), y=3×10⁻⁵e2.2×, R²=0.98)—circles represent individual currents measured at each E. coli concentration (n=3), the black horizontal line markers represent the mean of the current density measured at each concentration, and the error bars represent one standard deviation from the mean for each individual concentration; (e) square wave voltammograms in the presence of 10⁶ CFU/mL of E. coli or the non-specific bacteria P. aeruginosa, E. aerogenes, E. cloacae, and K. pneumoniae spiked in unprocessed urine sample (inset image: zoom-in of the non-specific bacteria data from the main figure); (f) corresponding peak current densities of bacterial loads in urine—circles represent individual currents measured for each bacterial sample, the bars represent the mean of the peak current density measured at each concentration, and the error bars represent one standard deviation from the mean for each concentration (n=3 independently prepared samples (c)-(d); n=6 and n=3 independently prepared samples for E. coli and other bacteria respectively (f)) (**p≤0.01, ***p≤0.001 calculated by two-tailed Student's t-test).

FIG. 10 shows selectivity of the integrated microgel magnetic bead assay for detecting E. coli in buffer in exemplary embodiments of the disclosure: (a) square wave voltammetry of the electrochemical signal from 10⁶ CFU/mL of E. coli (multiple scans represent n=6 chips) and the non-specific bacteria P. aeruginosa, E. aerogenes, E. cloacae, and K. pneumoniae spiked in buffer; (b) corresponding maximum current densities of bacterial loads in buffer—in (b), circles represent individual data points, and the error bar represents the standard deviation (n=3 and n=6 independently prepared samples for other bacteria and E. coli, respectively; **p≤0.01, ***p≤0.001 calculated by two-tailed Student's t-test).

FIG. 11 shows clinical validation of the E. coli mMB kit in exemplary embodiments of the disclosure: (a) E. coli mMB kit for UTI detection connected to a mobile operated handheld electrochemical reader; (b) square wave voltammograms of E. coli+ and E. coli− urine samples (inset image: zoom-in of the E. coli− data from the main figure); (c) peak current densities measured for the unprocessed urine of four independent patients belonging to the E. coli+/culture+ cohort, two independent patients belonging to the E. coli−/culture+ (E. faecalis positive) cohort, and two independent patients belonging to the E. coli−/culture− (healthy) cohort—circles represent individual currents measured for each bacterial sample, the bars represent the mean of the peak current density measured at each concentration, and the error bars represent one standard deviation from the mean for each concentration (n=6 repeats from a single clinical sample); **p≤0.05, ***p≤0.005, ***p≤0.001 calculated by two-tailed Student's t-test.

FIG. 12 shows storage stability of the E. coli assay components in exemplary embodiments of the disclosure: (a) current density (grey bar) and percentage change in signal relative to day 0 (black square) for the detection of an E. coli load of 10⁵ CFU/mL using e-Chips stored under vacuum sealed conditions at 4° C. for 5, 15, and 30 days; (b) storage stability of DNAzyme-mMB: i) current density obtained for an E. coli load of 105 CFU/mL using DNAzyme-mMBs stored in buffer under vacuum sealed conditions at 4° C. (light grey) or lyophilized and stored under vacuum sealed conditions at 4° C. (dark grey); ii) percentage change in the signal after 5, 15, and 30 days of storage (calculated with respect to the signal measured at day 0) for DNAzyme-mMBs stored in buffer (squares) and lyophilized DNAzyme-mMBs (diamonds)—inset indicates the percentage change in current density for the lyophilized DNAzyme-mMBs.

### DETAILED DESCRIPTION

### I. Definitions

Unless otherwise indicated, the definitions and embodiments described in this and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting.

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present. For greater clarity, the phrase “at least one of” is understood to be one or more. The phrase “at least one of . . . and . . . ” is understood to mean at least one of the elements listed or a combination thereof, if not explicitly listed. For example, “at least one of A, B, and C” is understood to mean A alone or B alone or C alone or a combination of A and B or a combination of A and C or a combination of B and C or a combination of A, B, and C.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

It will be understood that any component defined herein as being included may be explicitly excluded from the claimed invention by way of proviso or negative limitation, such as any specific compounds or method steps, whether implicitly or explicitly defined herein.

In addition, all ranges given herein include the end of the ranges and also any intermediate range points, whether explicitly stated or not.

The term “room temperature” as used herein refers to a temperature in the range of about 20° C. and about 25° C.

The term “sample” or “test sample” as used herein refers to any material in which the presence or amount of a target analyte is unknown and can be determined in an assay. The sample may be from any source, for example, any biological (e.g. human or animal samples, including clinical samples), environmental (e.g. water, soil or air) or natural (e.g. plants) source, or from any manufactured or synthetic source (e.g. food or drinks). The sample may be comprised or is suspected of comprising one or more analytes. The sample may be a “biological sample” comprising cellular and non-cellular material, including, but not limited to, tissue samples, saliva, sputum, urine, blood, serum, other bodily fluids and/or secretions.

The term “target”, “analyte” or “target analyte” as used herein refers to any agent, including, but not limited to, a small inorganic molecule, small organic molecule, metal ion, biomolecule, toxin, biopolymer (such as a nucleic acid, carbohydrate, lipid, peptide, protein), cell, tissue, microorganism, and virus, which one would like to sense or detect. The analyte may be either isolated from a natural source or synthetic. The analyte may be a single compound or a class of compounds, such as a class of compounds that share structural or functional features. The term analyte also includes combinations (e.g. mixtures) of compounds or agents such as, but not limited, to combinatorial libraries and samples from an organism or a natural environment.

The term “treatment or treating” as used herein refers to an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable.

The term “subject” as used herein includes all members of the animal kingdom including mammals such as a mouse, a rat, a dog and a human.

The term “microorganism” as used herein refers to a microscopic organism that comprises either a single cell or a cluster of single cells including, but not limited to, bacteria, fungi, archaea, protists, algae, plankton and planarian.

The term a “microorganism target” as used herein refers to a molecule, compound or substance that is present in or on a microorganism or is generated, excreted, secreted or metabolized by a microorganism.

The term “nucleic acid” as used herein refers to a biopolymer comprising monomers of nucleotides, such as deoxyribonucleic acid (DNA), ribonucleic acid (RNA) and other polynucleotides of modified nucleotides and/or nucleotide derivatives, and may be either double stranded (ds) or single stranded (ss). In some embodiments, modified nucleotides may contain one or more modified bases (e.g. unusual bases such as inosine, and functional modifications to the bases such as amino), modified backbones (e.g. peptide nucleic acid, PNA) and/or other chemically, enzymatically, or metabolically modified forms.

The term “catalytic nucleic acid”, “catalytic DNA”, “deoxyribozyme”, “DNA enzyme” or “DNAzyme” as used herein refers to a nucleic acid molecule or oligonucleotide sequence that can catalyze or initiate a reaction, optionally in response to specifically recognizing and binding to a target analyte. DNAzymes may be single-stranded DNA, and may include RNA, modified nucleotides and/or nucleotide derivatives.

The term “aptamer” as used herein may refer to a short, chemically synthesized nucleic acid molecule or oligonucleotide sequence which can be generated by in vitro selection to fold into specific three-dimensional (3D) structures that bind to a specific analyte with dissociation constants, for example, in the pico- to nano-molar range. Aptamers may be single-stranded DNA, and may include RNA, modified nucleotides and/or nucleotide derivatives. Aptamers may also be naturally occurring RNA aptamers termed “riboswitches”.

The term “antibody” as used herein may refer to a glycoprotein, or antigen-binding fragments thereof, that has specific binding affinity for an antigen as the target analyte. Antibodies may be monoclonal and/or polyclonal antibodies.

The term “hybridizes”, “hybridized” or “hybridization” as used herein refers to the sequence specific non-covalent binding interaction with a complementary, or partially complementary, nucleic acid sequence.

The term “capture probe” as used herein may refer to a probe that recognizes and binds, directly or indirectly, to a reporter moiety.

The term “reporter moiety” as used herein may refer to a moiety comprising a molecule (e.g. compound) for reporting the presence of an analyte. For example, the moiety is used for transducing the presence of an analyte recognized by the recognition moiety to a detectable signal.

The term “biorecognition agent” as used herein refers to a biological entity that acts as a molecular recognition element and is capable binding to a target analyte.

The term “microgel” as used herein refers to a particulate hydrogel with at least one dimension on the length scale of about 10 nm to about 1000 μm.

It will be understood that any component defined herein as being included may be explicitly excluded by way of proviso or negative limitation, such as any specific compounds or method steps, whether implicitly or explicitly defined herein.

### II. Compositions and Methods of the Disclosure

Disclosed herein, in embodiments, is the development of DNAzyme-functionalized microgel magnetic beads (mMBs) based on the protein repellent polymer poly(oligo(ethylene glycol) methacrylate (POEGMA) that can combine high density biofunctionalization, low biofouling, and rapid magnetic separation, enabling direct biosensing, such as bacterial detection in undiluted and unprocessed samples, such as undiluted and unprocessed urine, collected from symptomatic patients suspected of having an infection, such as, urinary tract infections (UTIs).

POEGMA is a hydrophilic and non-ionic mimic of poly(ethylene glycol) (PEG), the most widely used protein repellent polymer in biomedical applications due to its dual functionality of maintaining a strong hydration layer and sterically excluding bio-foulants from the microgel bead surface by virtue of its flexible hydrophilic chains; however, unlike PEG, POEGMA can be easily (co-)polymerized to create functional hydrogels or microgels that can meet most of the key functional requirements for POC microbeads (i.e. easy functionalization, high degree of hydration, and suppressed biofouling). By physically encapsulating superparamagnetic iron oxide nanoparticles (SPIONs) into micron-sized POEGMA-based microgel beads and functionalizing the resulting magnetic microgel beads with DNAzymes programmed to generate an electrochemical signal in response to specific bacterial targets, in embodiments, a sensitive, specific, rapid biosensing assay platform has been developed. This assay can be used, for example, as a point-of-care platform that can detect and identify bacterial infections directly in complex biological fluids using two simple steps and without any requirement for sample pre-processing or the addition of reagents.

In some embodiments, the microgel magnetic bead assay enables highly efficient conjugation and hydration of the immobilized DNAzymes, resulting in low limits-of-detection with high specificity against multiple urinary pathogens. For example, the limits-of-detection for a target analyte in buffer can be from about 1 CFU/mL to about 20 CFU/mL; about 5 CFU/mL to about 15 CFU/mL; about 5 CFU/mL to about 10 CFU/mL; about 6 CFU/mL to about 8 CFU/mL; or about 6 CFU/mL in buffer. In embodiments, the target analyte is detected in the sample in an amount of about 10 CFU/mL to about 10⁶ CFU/mL; about 10 CFU/mL to about 10⁵ CFU/mL; about 10 CFU/mL to about 10⁴ CFU/mL; about 10 CFU/mL to about 10³ CFU/mL; about 10 CFU/mL to about 500 CFU/mL; about 50 CFU/mL to about 200 CFU/mL; about 50 CFU/mL to about 175 CFU/mL; about 100 CFU/mL to about 150 CFU/mL; about 125 CFU/mL to about 150 CFU/mL; about 130 CFU/mL to about 145 CFU/mL; about 135 CFU/mL to about 145 CFU/mL; about 135 CFU/mL to about 140 CFU/mL; or about 138 CFU/mL. In embodiments, the sample is unprocessed urine. In some embodiments, the limits-of-detection for a target analyte in unprocessed urine can be from about 50 CFU/mL to about 200 CFU/mL; about 50 CFU/mL to about 175 CFU/mL; about 100 CFU/mL to about 150 CFU/mL; about 125 CFU/mL to about 150 CFU/mL; about 130 CFU/mL to about 145 CFU/mL; about 135 CFU/mL to about 145 CFU/mL; about 135 CFU/mL to about 140 CFU/mL; or about 138 CFU/mL in unprocessed urine. The assay can be performed within about 30 minutes to about 10 hours; about 30 minutes to about 8 hours; about 30 minutes to about 7 hours; about 30 minutes to about 6 hours; about 30 minutes to about 5 hours; about 30 minutes to about 4 hours; about 30 minutes to about 3 hours; about 30 minutes to about 2 hours; about 30 minutes to about 1 hour; about 45 minutes to about 1 hour; or about 1 hour. The assay of the disclosure can be used to identify which patients are infected with, for example, E. coli as the causative organism for their UTI symptoms.

Accordingly, in embodiments, provided is a magnetic microparticle comprising a magnetic nanoparticle encapsulated by a polymer hydrogel. In embodiments, the polymer hydrogel comprises a three-dimensional crosslinked network of water-soluble polymer(s). In embodiments, the polymer hydrogel comprises a protein repellent polymer. In embodiments, the polymer hydrogel comprises may include, for example, poly(oligo(ethylene glycol) methacrylate or other poly(ethylene glycol) derivatives. In other embodiments, the polymer hydrogel comprises a zwitterionic polymer, including but not limited to, polysulfobetaine(s), poly(sulfobetaine) methacrylate, polycarboxybetaine(s), poly(carboxybetaine) methacrylate, poly(phosporylcholine). In other embodiments, the hydrogel polymer comprises poly(N-vinylpyrrolidone), poly(acrylamide)s, polyglycidols, poly(2-oxazoline)s, or derivatives thereof. In specific embodiments, the polymer hydrogel comprises poly(oligo(ethylene glycol) methacrylate (POEGMA). In some embodiments, the magnetic microparticle is fabricated by copolymerizing oligo(ethylene glycol methacrylate), methacrylic acid and ethylene dimethacrylate in the presence of a magnetic nanoparticle. In other embodiments, the magnetic nanoparticle is grown inside a pre-formed microgel.

In some embodiments, the microparticle is a microgel. In some embodiments, the microgel comprises at least one dimension on the length scale of about 10 nm to about 1000 μm; about 50 nm to about 100 μm; about 100 nm to about 10 μm; about 1 μm to about 10 μm; about 1 μm to about 9 μm; about 1 μm to about 8 μm; about 3 μm to about 7 μm; about 4 μm to about 6 μm; or about 5 μm. In some embodiments, the microgel comprises at least one dimension on the length scale of at least about 5 μm.

In some embodiments, the magnetic nanoparticle comprises iron, cobalt, nickel, or rare earth elements. In specific embodiments, the magnetic nanoparticles comprise iron oxide. In some embodiments, the magnetic nanoparticle comprises superparamagnetic iron oxide nanoparticles (SPIONs).

In some embodiments, the microparticle is from about 0.5 μm to about 100 μm; about 0.5 μm to about 75 μm; about 0.5 μm to about 50 μm; about 0.5 μm to about 30 μm; about 0.5 μm to about 20 μm; about 1 μm to about 15 μm; about 2 μm to about 10 μm; about 3 μm to about 9 μm; about 4 μm to about 8 μm; about 4 μm to about 6 μm; or about 5 μm. In some embodiments, the microparticle is at least about 5 μm in diameter.

In some embodiments, the microparticle is prepared by microfluidics, suspension polymerization, emulsion polymerization, membrane templating, or precipitation polymerization. In some embodiments, the microparticle is prepared by inverse emulsion templating. In some embodiments, the microparticle is prepared via a semi-batch inverse suspension polymerization strategy. In some embodiments, the microparticle is prepared by precipitation, coacervation, microfluidics processes, air jetting, spray drying, or electrospray techniques.

In some embodiments, the microparticle further comprises a biorecognition agent functionalized on and/or in the microparticle. In some embodiments, carboxylic acid groups from the methacrylic acid residues are grafted with an amine-terminated biorecognition agent using carbodiimide chemistry.

In some embodiments, the biorecognition agent is at least one of an antibody, an aptamer, and a DNAzyme. In some embodiments, the biorecognition agent is a DNAzyme. In some embodiments, the DNAzyme is “RNA-cleaving” and catalyzes the cleavage of a particular substrate, for example a nucleic acid sequence comprising one or more ribonucleotides, at a defined cleavage site. In some embodiments, the DNAzyme cleaves a single ribonucleotide linkage. In some embodiments, the single ribonucleotide linkage is in a nucleic acid sequence wherein the remaining nucleotides are ribonucleotides. In some embodiments, the single ribonucleotide linkage is in a nucleic acid sequence wherein the remaining nucleotides are deoxyribonucleotides. In some embodiments, the DNAzyme cleaves a nucleic acid sequence at a single ribonucleotide linkage thereby producing a nucleic acid cleavage fragment.

Provided herein is also an assay for detecting the presence of a target analyte in a sample comprising:
a) the magnetic microparticle disclosed herein, wherein the biorecognition agent further comprises a reporter moiety;
b) an electrochemical chip comprising a working electrode, a counter electrode and a reference electrode; and
c) a capture probe functionalized on the working electrode;
wherein binding of the biorecognition agent to the target analyte results in production of an electrochemical, electroluminescent or photoelectrochemical signal.

In some embodiments, the signal is an electrochemical signal. In some embodiments, the electrochemical signal is measured by amperometry, voltammetry, photoelectrochemistry, electrochemiluminescence, potentiometry or impedance. In some embodiments, the electrochemical signal is measured by square wave voltammetry.

In some embodiments, the working electrode comprises a conductive material, semi-conductive material, or a combination thereof. In some embodiments, the working electrode comprises metal, metal alloy, metal oxide, superconductor, semi-conductor, carbon-based material, conductive polymer, or combinations thereof. Examples include, but are not limited to, gold, platinum, palladium, carbon-based materials such as glassy carbon, graphite, graphene, or carbon nanotubes, nickel oxide, bismuth oxide, indium tin oxide, and titanium dioxide.

In some embodiments, the working electrode comprises metal. The metal may be selected from aluminum (Al), antimony (Sb), bismuth (Bi), boron (B), cadmium (Cd), carbon (C), cerium (Ce), chromium (Cr), cobalt (Co), copper (Cu), dysprosium (Dy), erbium (Er), europium (Eu), gadolinium (Gd), germanium (Ge), gold (Au), graphite (C), hafnium (Hf), holmium (Ho), indium (In), iridium (Ir), iron (Fe), lanthanum (La), lutetium (Lu), magnesium (Mg), manganese (Mn), molybdenum (Mo), neodymium (Nd), nickel (Ni), niobium (Nb), palladium (Pd), platinum (Pt), praseodymium (Pr), rhenium (Re), ruthenium (Ru), samarium (Sm), selenium (Se), scandium (Sc), silver (Ag), silicon (Si), tantalum (Ta), terbium (Tb), thulium (Tm), tin (Sn), titanium (Ti), tungsten (W), vanadium (V), ytterbium (Yb), yttrium (Y), zirconium (Zr) and/or zinc (Zn). Typically, the metals are selected from gold, other noble metals, or combinations thereof.

In some embodiments, the working electrode comprises gold.

In some embodiments, the counter electrode is both the counter electrode and the reference electrode.

In some embodiments, the working electrode further comprises hierarchical structures. The electrodes may be made from any suitable method, for example, a seed layer for the hierarchically structured electrodes may be made by sputter-coating, evaporation, chemical vapor deposition, or a pulsed laser method, ink jet printing.

In some embodiments, the working electrode further comprises an anti-fouling coating. In some embodiments, the coating comprises mercaptohexanol (MCH).

In some embodiments, the biorecognition agent is at least one of an antibody, an aptamer, and a DNAzyme. In some embodiments, the biorecognition agent is a DNAzyme. In some embodiment, the DNAzyme recognizes a target analyte and cleaves a nucleic acid sequence at a single ribonucleotide linkage upon interaction of the DNAzyme with the target thereby releasing the reporter moiety as the cleavage fragment.

In an embodiment, the reporter moiety a redox, photoelectrochemical, passivating, semi-conductive and/or conductive species. In some embodiments, the reporter moiety comprises a redox species, a photoactive species, or a electrochemiluminescence species. In embodiments, the reporter moiety comprises the redox species. Examples of redox species include, but are not limited to, ruthenium haxaamine chloride, 3,7-Bis-[(2-Ammoniumethyl) (methyl)amino]phenothiazin-5-ium trifluoroacetate; 3,7-Bis-(piperazin-4-ium-1-yl)phenothiazin-5-ium trifluoroacetate; 3,7-Bis-[(2-ammoniumethyl)(methyl)amino]phenothiazin-5-ium chloride; and 3,7-Bis-(piperazin-4-ium-1-yl)phenothiazin-5-ium chloride, methylene blue, methylene blue succinimide, methylene blue maleimide, Atto MB2 maleimide (Sigma Aldrich) and other methylene blue derivatives, ferrocene and Fe⁺² and/or Fe⁺³ ions.

In some embodiments, the redox species is methylene blue.

In some embodiments, the reporter moiety comprises a biopolymer modified with the redox species. In some embodiments, the biopolymer comprises a nucleic acid. In some embodiments, the biopolymer comprises single-stranded DNA.

In an embodiment, the capture probe comprises a biopolymer. In an embodiment, the capture probe comprises a nucleic acid. In some embodiments, the capture probe comprises single-stranded DNA.

In an embodiment, the target analyte comprises a microorganism target. In some embodiments, the microorganism target is present in the extracellular matrix of a microorganism. In some embodiments, the microorganism target is present in the intracellular matrix of a microorganism. In some embodiments, the microorganism target comprises a protein, a nucleic acid, a small molecule, extracellular matrix, intracellular matrix, a cell of the microorganism, or any combination thereof. In some embodiments, the microorganism target is a crude or purified extracellular matrix or a crude or purified intracellular matrix. In some embodiments, the microorganism target is specific to a particular species or strain of microorganism.

In some embodiments, the microorganism is a bacterium. In some embodiments, the microorganism is a gram-negative bacterium, for example Escherichia coli, Salmonella typhimurium, Pseudomonas peli, Brevundimonas diminuta, Hafnia alvei, Yersinia ruckeri, Ochrobactrum grignonese, Achromobacter xylosoxidans, Moraxella osloensis, Acinetobacter lwoffi, and Serratia fonticola. In an embodiment, the microorganism is a gram-positive bacterium, for example Listeria monocytogenes, Bacillus subtilis, Clostridium difficile, Actinomyces orientalis, Pediococcus acidilactici, Leuconostoc mesenteroides, and Lactobacillus planturum. In some embodiment, the microorganism is a pathogenic bacterium (for example, a bacterium that causes bacterial infection), such as Escherichia coli O157:H7, Listeria monocytogenes, Salmonella typhimurium or Clostridium difficile.

In some embodiments, the microorganism is Escherichia coli.

In some embodiments, the sample is a urine sample. In some embodiments, the urine sample is an unprocessed urine sample. In some embodiments, the sample is a biological sample from a subject suspected of having an infection. In some embodiment, the sample is a biological sample from a subject suspected of having a urinary tract infection. In some embodiments, the biological sample is a sample of urine from the subject.

In some embodiments, the assay is for use in screening and/or diagnostics, treatment monitoring, environmental monitoring, health monitoring, and/or pharmaceutical development. In some embodiments, the biosensor is for use in screening, diagnostics, and/or health monitoring. In some embodiments, the assay is a point-of-care test. In some embodiments, the assay described herein is for use in detecting infection-causing pathogens in point-of-care diagnostics and health monitoring.

In some embodiments, the assay detects a urinary tract infection in a subject.

Also provided herein is a kit for detecting the presence of a target analyte in a sample, wherein the kit comprises
a) the magnetic microparticle disclosed herein, wherein the biorecognition agent further comprises a reporter moiety;
b) an electrochemical chip comprising a working electrode, a counter electrode and a reference electrode;
c) a capture probe functionalized on the working electrode;
d) a magnet; and
e) instructions for use of the kit.
   In some embodiments, the magnet is encased in a 3D printed tube holder.

In some embodiments, the kit further comprises a sample container. In some embodiments, the kit further comprises an electrical reader. In some embodiments, the kit further comprises a sample container and an electrical reader. In some embodiments, the sample container is a test tube or vial. In some embodiments, the reader is a hand-held device. In some embodiments, the kit further comprises reagents and/or solutions, such as buffers, to provide conditions for binding the biorecognition agent to the target analyte. In some embodiments, the kit further comprises an assay holder, wherein the holder comprises an incubation slot, a magnetic separation slot, and an electrochemical chip slot.

In some embodiments, the assay and/or kit described herein may be used without the need for sample pre-treatment, target labeling, and/or amplification. In some embodiments, the assay and/or kit may increase the accuracy and decrease the timeline for diagnosis.

Provided herein is also a method of determining the presence of a target analyte in a sample comprising:
a) exposing the magnetic microparticle of the assay disclosed herein to the sample to release the reporter moiety from the biorecognition agent in the presence of the target analyte;
b) separating the magnetic microparticle from the sample; and
c) depositing the sample of step b) to the electrochemical chip of the assay disclosed herein;
wherein the capture probe of the assay disclosed herein binds the reporter moiety to produce an electrochemical signal.

In some embodiments, the electrochemical signal is measured by square wave voltammetry.

In some embodiments, the magnetic microparticle is exposed to the sample under conditions for binding the biorecognition agent to the target analyte. In some embodiments, exposing the magnetic microparticle to the sample comprises incubating the magnetic microparticle with the sample for about 30 minutes.

In some embodiments, separating the magnetic microparticle from the sample comprises exposing the magnetic microparticle to a magnet for about 5 minutes.

In some embodiments, the method further comprises incubating the sample from step b) with the electrochemical chip for about 30 minutes after step c). In some embodiments, incubating the sample from step b) with the electrochemical chip is performed at about 37° C.

In some embodiments, the biorecognition agent is at least one of an antibody, an aptamer, and a DNAzyme. In some embodiments, the biorecognition agent is a DNAzyme.

In some embodiments, the target analyte comprises a microorganism target.

In some embodiments, the microorganism is Escherichia coi.

In some embodiments, the sample is a urine sample.

In some embodiments, the method detects a urinary tract infection in a subject. In some embodiments, the method further comprises a method of diagnosing a urinary tract in a subject. In some embodiments, the method further comprises a method of treating a urinary tract in a subject.

Also provided herein is use of the magnetic microparticles, the assay or the kit disclosed herein, to determine the presence of a target analyte. Also provided herein is the use of the magnetic microparticles to capture the target analyte. In some embodiments, the target analyte comprises Escherichia coli. In some embodiments, the magnetic microparticles, the assay or the kit disclosed herein is for use in detecting Escherichia coli. In some embodiments, the magnetic microparticles, the assay or the kit disclosed herein is for use in detecting a urinary tract infection in a subject.

### EXAMPLES

The following non-limiting examples are illustrative of the present disclosure:

### Methods

Materials and reagents: Oligo(ethylene glycol) methyl ether methacrylate (OEGMA, Mₙ˜500), methacrylic acid (MAA, 99%), ethylene glycol dimethacrylate (EGDMA, 98%), ammonium persulfate (KPS, ≥99.0%), iron(III) chloride hexahydrate (97%), iron(II) chloride tetrahydrate (98%), ammonium hydroxide solution (≥99.0%), sorbitan oleate (SPAN® 80), polysorbate 80 (TWEEN® 80), phosphate buffer solution (about 1.0 M, about pH 7.4), sodium chloride (NaCl, ≥99.0%), magnesium chloride (MgCl₂, ≥99.0%), 6-mercapto-1-hexanol (MCH, ≥99%), tris(2-carboxyethyl)phosphine hydrochloride (TCEP), potassium hexacyanoferrate(II) trihydrate ([Fe(CN)₆]⁴—, ≥99.95%), gold(III) chloride solution (HAuCl₄, 99.99%), N-(3-dimethylaminopropyl)-N′-ethylcarbodiimide hydrochloride (EDC, ≥99.0%), N-hydroxysuccinimide (NHS, ≥98.0%), bovine serum albumin (≥96%, lyophilized powder), IgG from human serum (reagent grade, ≥95%, lyophilized powder), and fibrinogen from human plasma (about 50% to about 70% protein) were purchased from Sigma-Aldrich (Oakville, Canada) and used as received. Inhibitors in the OEGMA monomer (200 ppm BHT and 100 ppm MEHQ) were removed using an alumina oxide (Al₂O₃)-filled vertical glass column. Alumina oxide was purchased from Thermo Fisher Scientific (USA). N-hexane (95%), paraffin (95%), sulfuric acid (H₂SO₄, 98%) and 2-propanol (99.5%) were purchased from Caledon Laboratories (Georgetown, Canada). Standard Pierce 660 nm colorimetric assay reagent was purchased from Thermo Fisher Scientific (USA). Methylene blue (MB)-labelled DNA and non-MB labelled DNA were obtained from Biosearch Inc. and Integrated DNA Technologies (IDT) respectively. Hydrochloric acid (HCl; about 37% w/w) was purchased from LabChem (Zelienople, PA). Commercial Dynabeads™ Myone™ Carboxylic acid-65011 (1 μm average diameter) were used for comparison. All water used was of Milli-Q grade (ddH₂O, resistivity >18 mΩ).

Superparamagnetic iron oxide nanoparticle (SPION) synthesis: SPIONs were prepared using the co-precipitation method. Iron(III) chloride hexahydrate (about 3.04 g) and iron(II) chloride tetrahydrate (about 1.98 g) were dissolved at an about 2:1 molar ratio in about 12.5 mL ddH₂O. The solution was purged with nitrogen for about 10 minutes, after which about 6.5 mL ammonium hydroxide was added dropwise under magnetic stirring at about 800 rpm while maintaining nitrogen purging. The resulting SPIONs were separated using a magnet over at least five purification cycles with ddH₂O, with the final product stored in ddH₂O at room temperature. The concentration of SPIONs was determined by comparing the mass of SPIONs before and after drying in an about 80° C. oven overnight.

Magnetic microgel bead (mMB) synthesis: Magnetic poly(ethylene glycol) methyl ether methacrylate (POEGMA)-based microgel magnetic beads were synthesized using an inverse emulsion templating method. The continuous phase was prepared by mixing about 42 mL of paraffin oil and about 1.5 mL of an about 75:25 vol % surfactant mixture of Span 80 and Tween 80 and was heated to about 65° C. under about 500 rpm mechanical stirring and a nitrogen purge for about 20 minutes. The dispersed phase was prepared by dissolving about 2 g OEGMA500 (inhibitors removed), about 200 mg EGDMA, and about 780 mg MAA (methacrylic acid) in about 5 mL of distilled deionized water. Pre-synthesized SPIONS were dispersed at a concentration of about 1.5 wt % inside the monomer solution via bath sonication for about 10 minutes, after which the pH of the dispersed phase was adjusted to about 7. A three-stage semi-batch process was then used to prepare the magnetic microgels: (Stage 1) about 100 μL of initiator solution (prepared by dissolving about 60 mg KPS in about 600 μL water), about 2 mL of the dispersed phase, and about 500 μL of surfactant were added to continuous phase dropwise; (Stage 2) Starting about 20 minutes after stage 1 was complete, about 250 μL of initiator solution, about 2 mL of the dispersed phase, and about 250 μL of surfactant were added dropwise to the continuous phase; (Stage 3) Starting about 20 minutes after stage 2 was complete, about 250 μL of initiator solution, about 2 mL of the dispersed phase, and about 250 μL of surfactant were added to the continuous phase. The reaction was left to proceed for about 2 hours at about 65° C. under nitrogen purging and about 500 rpm stirring. Following, the surfactants and continuous phase were extracted with hexane, after which the collected magnetic beads (in the aqueous phase) were magnetically washed with water over 5 cycles and stored at about 4° C. in water. mMB size and size distribution were characterized using laser diffraction (Mastersizer 2000, Malvern Instruments), while electrophoretic mobility was assessed using a Brookhaven 90Plus instrument. The MAA content of the mMBs was assessed using conductometric base-into-acid titration. The internal morphology and SPION distribution of the mMBs were characterized using scanning electron microscopy, while the total SPION content in the beads was measured by thermal gravimetric analysis. Targeted mMBs were fabricated by coupling amino-terminated DNAzymes to —COOH groups from the MAA residues in the mMB, with the degree of DNAzyme incorporation measured using fluorescently-labelled DNAzyme to quantify the percentage of DNAzymes bound to the mMB phase.

Magnetic Microgel Bead (mMB) Characterization:

Particle size: The particle size and particle size distribution of the magnetic microgel beads (mMBs) were characterized using laser diffraction (Mastersizer 2000, Malvern Panalytical). Each sample was measured three times, with the average size based on particle surface area reported.

Electrophoretic mobility: The electrophoretic mobility was measured using a Brookhaven 90Plus zeta potential analyzer operating in phase analysis light scattering (PALS) mode. mMBs were magnetically washed and re-suspended in about 0.1 M NaCl. Electrophoretic mobility measurements were performed at a count rate ranging from about 300 kilocounts/s to about 700 kilocounts/s, with the average of six measurements reported for each sample.

Chemistry: The methacrylic acid content of the mMBs was determined by conductometric base-into-acid titration using a Burivar-I2 automatic buret (Mantech) running PC titrate software. About 10 to about 15 mg of magnetic microgel suspended in ddH₂O was magnetically washed with about 3 mM NaCl over three cycles and resuspended in about 3 nM NaCl solution. The mMB dispersion was purged with nitrogen for about 20 minutes prior to titration, and the pH of the mMB suspension was adjusted to about 2.75. The system was set to inject about 0.001 mL 0.1 M NaOH every about 20 seconds until the suspension achieved a pH of about 11. The degree of MAA functionalization was calculated based on the amount of NaOH used to titrate the carboxyl groups.

Dispersion and magnetic separation: mMB dispersion was assessed using inverted brightfield microscopy (Olympus) by applying a single drop of the mMB suspension on a glass slide and observing the suspension directly with different objectives (10×, 20×, 40×). The magnetic separability of the mMBs was assessed by bringing a homogeneously dispersed mMB suspension in about a 20 mL scintillation vial in contact on one side with a neodymium magnet (40-42 MG-Oe, 318-342 kJ/m³). The time between the first exposure to the magnet and the complete isolation of magnetic beads from the suspension (as indicated by the change in color in the suspension from brown to clear) was recorded as the separation time.

Morphology The distribution of SPIONs inside mMBs was assessed using scanning electron microscopy (SEM, Tescan) using an operating voltage of about 10 kV under low vacuum mode. Samples were prepared by dropping about 0.5 mL of an about 1 mg/mL mMB suspension on a SEM stub covered with carbon tape and drying the sample at room temperature prior to imaging.

SPION content: Thermogravimetry (Mettler Toledo TGA/DSC 3+) was used to determine the content of SPIONs inside the mMBs. The fabricated mMB suspension was freeze dried to remove water, after which between about 1 to about 5 mg of dried sample was loaded into a pre-weighed alux 70 μL crucible. Three stages of heating, all conducted under a flow of about 30 mL/min of argon, were used: (1) heating from about 25° C. to about 100° C. at about 20 K/min; (2) holding at about 100° C. for about 5 minutes; and (3) further raising the temperature from about 100° C. to about 800° C. at about 10 K/min. The mass was continuously monitored over time, with the dry SPION content of the magnetic microgels calculated by dividing the percentage of sample mass remaining after thermal decomposition by the total mass of sample.

Colloidal stability: The long-term stability of mMBs was tested by continuously monitoring the electrophoretic mobility and particle size/size distribution of three different batches of mMBs over a period of one month, using the same techniques described above.

DNAzyme immobilization on mMBs: Functional mMBs were prepared by carbodiimide-mediated grafting of an amino-terminated DNAzyme, the truncated version of the 6-carboxyfluorescein (FAM)-labelled DNAzyme used elsewhere.¹ The sequence is provided in Table 1. About 1 mg/mL of mMBs were washed in about 25 mM of MES buffer. The washed mMBs were then activated in EDC:NHS:MES (about 10 mM:10 mM:25 mM) at about pH 6.5 for about 30 minutes at room temperature. Upon further washing in about 25 mM MES buffer, about 1 μM amine terminated DNAzyme was added, and the suspension was incubated for about 12 to about 18 hours at about 4° C. in dark. The resulting conjugated mMB-DNAzyme were then washed in about 25 mM MES and 1×PBS (about pH 7.1) and suspended in 1×PBS for further use in the assay.

### Fluorescence Characterization of Immobilized DNAzyme Properties:

Fluorescence assay to quantify DNAzyme loading and cleavage efficiency: Amine-terminated RNA cleaving DNAzyme was prepared by T4 DNA ligase-mediated DNA ligation of a fluorescent substrate and DNAzyme in the presence of a ligation template. The fluorescein signal was quantified using a plate reader (Tecan M200) operating at an excitation wavelength of about 488 nm and an emission wavelength of about 520 nm.

DNAzyme loading efficiency: For the fluorescence detection of bacteria in buffer, bacterial target dilutions in 1×PBS (about 20 μL) were added to an about 1 mg/mL mMB-DNAzyme suspension (about 5 μL) and incubated for about 30 min at room temperature. Following, the mMBs were magnetically separated (about 5 minutes) and the concentration of residual DNAzymes in the supernatant was measured using the same fluorescence technique described above; the difference between the initial DNAzyme concentration and the residual solution concentration following grafting was used to quantify the amount of the DNAzymes immobilized on mMBs.

DNAzyme cleavage efficiency: To assess the activity of the mMB-immobilized DNAzymes, about 5 μg of the functionalized mMBs was mixed with about 20 μL of E. coli targets at a concentration of about 10⁶ CFU/mL in PBS buffer for about 2 hours at room temperature under gentle shaking. Magnetic beads were then washed with 1×PBS (about pH 7.4) and removed from the suspension magnetically, with the cleaved DNA probe concentration in the supernatant measured via the same fluorescence protocol described above.

Electrochemical chip fabrication: The electrochemical chip was fabricated on polystyrene (PS) sheets (Graphix Shrink Film, Graphix, Maple Heights, Ohio). The PS was cleaned by rinsing in ethanol and ddH₂O followed by N₂ drying. Following, the PS was masked with the vinyl sheet (FDC 4304, FDC graphic films, South Bend, Indiana). The chip pattern was designed in Adobe Illustrator, applied on the vinyl sheet, and cut into the desired pattern using a Robo Pro CE5000-40-CRP cutter (Graphtec America Inc., Irvine, CA). The patterns were then exposed by peeling the cut vinyl (keeping the rest of the area masked) and applying an about 100 nm thick gold (Au) film using DC sputtering (MagSput™, Torr International), after which the vinyl mask was removed. The Au sputtered chip was comprised of three electrodes: An Au working electrode (WE), an Au counter electrode (CE), and an Au reference electrode (RE). The WE were rinsed in isopropanol (IPA) and ddH₂O followed by nano structuring with gold hierarchical structures by electrodeposition in a solution of about 10 mM gold chloride (HAuCl₄) and about 0.5 mM HCl using a CHI 420B potentiostat (CH Instruments, Austin, TX), with deposition conducted under potentiostat conditions of about −0.6 V (anodic negative) for about 600 s using Ag/AgCl as the reference and Pt wire as the counter electrode.

Electrochemical mMB assay platform fabrication: The mMB assay platform consisted of a 3-D printed polyvinyl chloride (about 5 cm×about 3 cm×about 2 cm) support printed using an Original Prusa i3 MK3S 3D printer, a tube holder for the mMB tube, a magnet holder hole to securely contain the magnet upon which the tube sits, a chip holder to insert the electrochemical chip, and a reservoir to retain the detection solution on the chip. The gold micro/nanostructured electrochemical chip was fabricated by electrodeposition. The thiol-functionalized capture probe was immobilized on the working electrode via self-assembly followed by the blocking of unbound sites using about 100 mM 6-mercaptohexanol (MCH). To immobilize the complementary capture probe on the working electrode surface for binding the released mMB-labelled DNA barcode upon bacteria-induced cleavage, the WE were rinsed with isopropyl alcohol and ddH₂O followed by electrochemical activation using cyclic voltammetry in about 0.1 M H₂SO₄ (potential range: about 0 to about 1.5 V, scan rate: about 0.1 V, cycles: about 40). An about 1p M single stranded thiol terminated capture probe (CP) solution (about 3 μL) was reduced with about 100 μM tris(2-carboxyethyl) phosphine (TCEP) for about two hours in the dark at room temperature and then deposited on the electrochemically activated WE for about 18 hours. Following CP deposition, about 3 μL of an about 100 mM 6-mercaptohexanol (MCH) solution was deposited as backfill on the surface for about 20 minutes in the dark at room temperature to block any uncoated sites on the gold electrode surface.

Bacterial target preparation: Escherichia coli K12 (E. coli K12; MG1655) was streaked out on a LB agar plate aerobically at about 37° C. for about 24 hours to obtain single colonies. A single colony was inoculated in about 5 mL liquid LB for about 8-hour aerobic culture at about 37° C., about 250 rpm to reach OD˜1. The bacteria target was prepared as follows: about 1 mL of each bacterial culture (about 10⁸ CFU/mL) was centrifuged at about 10000×g for about 10 min and the clear supernatant was discarded. The pellet was resuspended in about 500 μL of 1×reaction buffer (about 50 mM HEPES, about pH 7.5, about 150 mM NaCl, about 15 mM MgCl₂, about 0.01% Tween 20), after which the cell suspension was heated at about 90° C. for about 5 min and left at room temperature for another about 10 min. After centrifugation at about 15000×g for about 10 min, the clear supernatant was collected as the bacterial target. Bacterial targets for Klebsiella pneumoniae, Enterobacter aerogenes, Enterobacter cloacae, and Pseudomonas aeruginosa were prepared using the same protocol.

### Electrochemical Characterization:

Electrochemically active surface area measurement: The electrochemically active surface area of the Au nanostructured WE was calculated by performing cyclic voltammetry (CHI 420B, Austin, TX) in about 0.1 M H₂SO₄ (potential range: 0-1.5 V, scan rate: about 0.1 V, cycles: about 40). The area under the reduction peak was integrated to calculate the electrochemical charge involved in the redox process, which was subsequently divided by the surface charge density involved in forming a monolayer of AuOₓ (about 482 μC/cm²).

Reproducibility study: Three independently fabricated electrochemical chips were validated post-cleaning, post-probe addition, and post-MCH deposition using cyclic voltammetry (CHI 420B, Austin, TX) in a about 2 mM potassium hexacyanoferrate (II) solution (potential range: 0-0.5 V, scan rate: about 0.1 V, cycles: about 2). A positive control with an E. coli load of about 10⁵ CFU/mL in PMT 20 (phosphate buffer (about 25 mM): NaCl (about 25 mM): MgCl₂ (about 100 mM): Tween 20 (about 0.001%)) was detected on each e-Chip to assess the reproducibility of the assay. Three different batches of DNAzyme-mMB were also tested using the same positive control E. coli load of about 10⁵ CFU/mL in PMT 20.

Bacterial detection in buffer: For the electrochemical detection of bacteria in buffer, bacterial target dilutions made in PMT 20 (phosphate buffer (about 25 mM): NaCl (about 25 mM): MgCl₂ (about 100 mM): Tween 20 (about 0.001%) (about 5 μL) were added to an about 1 mg/mL mMB-DNAzyme suspension (about 5 μL) and incubated for about 30 min at room temperature. Following, the mMBs were magnetically separated (about 5 minutes) and the supernatant (about 9 μL) was recovered and applied as the detection solution to the electrochemical chip (30 min incubation time at about 37° C.). The methylene blue reduction signal of the hybridized MB-barcode with capture probe was measured by square wave voltammetry (SWV, CHI 420B, Austin, TX) over a voltage range of about 0 V to about −0.6 V (anodic negative).

Sensitivity assessment: The sensitivity of the mMB assay was assessed using bacterial target dilutions in PMT-20 (PBS—about 25 mM, NaCl—about 25 mM, MgCl₂—about 100 mM, Tween 20—about 0.001%) by adding bacterial target dilutions to DNAzyme-grafted mMB suspensions (about 1 mg/mL) at a about 1:1 volume ratio and incubating for about 30 min at room temperature. Following, mMBs were magnetically separated (about 5 minutes), and the supernatant (about 10 μL) was applied on the e-Chip for about 30 min at about 37° C. The methylene blue reduction signal of the released and subsequently captured MB-barcode was measured by square wave voltammetry. Urine samples were stored at about 4° C. and tested within about 5 days by diluting the bacterial target in undiluted urine following the same protocol as described above for the PMT-20.

Bacterial detection in urine: All urine samples were acquired and handled according to the protocols approved by the Hamilton Integrated Ethics Board (HiREB). Urine samples were first assessed for their bacterial concentration using the culture on the Walk Away Specimen Processor (WASP) and then collected from the Hamilton General's Clinical Pathology lab. The urine samples were stored at about 4° C. and tested within about 5 days of collection using the mMB integrated electrochemical assay. The detection of the bacteria spiked in urine was carried out by diluting the bacterial CIM in undiluted urine, after which the assay was conducted using the workflow described above for the buffer tests.

Kinetics study in urine: Assessment of the kinetics of the DNAzyme interaction with the E. coli target (causing the cleavage of the redox DNA barcode) and the capture of the redox DNA barcode on the e-Chip (enabling detection) was performed by spiking about 1000 CFU/mL of the E. coli target into unprocessed urine followed by incubation over different defined times (about 5, about 15, about 30, about 45, or about 60 minutes) using a Sensit smart potentiostat (PalmSens BV, Netherlands).

Specificity assessment: Assay specificity was assessed by spiking a panel of gram-negative urinary pathogens (Klebsiella pneumoniae, Enterobacter aerogenes, Enterobacter cloacae, and Pseudomonas aeruginosa) into either buffer or healthy urine using the CHI 420B potentiostat. The same dilutions and assay protocol were used for detection as described previously for buffer and urine spiked with E. coli.

Clinical sample assessment: Clinical assessment was performed based on eight UTI patient urine samples collected following the protocols used for acquiring urine for the bacterial spike experiment, including 4 E. coli+/culture+(>10² CFU/mL) and 4 E. c-li− (2 culture—(no bacterial growth) and 2 culture+ (E. faecalis) growth—identified via culture) samples. The clinical urine samples were mixed with the mMB-DNAzyme suspension (about 1 mg/mL) using a volume ratio of about 2:1 and incubated for about 30 minutes in room temperature. Following, mMBs were removed via magnetic separation (about 5 min) and the recovered supernatant (about 10 μL) was dropped on the electrochemical chip for about 30 minutes at about 37° C. The methylene blue reduction signal of the MB-barcode hybridized with capture probe was measured by square wave voltammetry (Sensit smart, PalmSens BV, Netherlands) over a voltage range of about 0 V to about −0.6 V (anodic negative). The limit-of-blank (LOB, defined as the highest signal obtained in response to a solution that is void of target analyte and accounting for any non-specific cleavage of the DNAzyme by native DNAse/RNAse in the biological samples) was calculated from the mean and the standard deviation of the signal obtained from the blank sample (LOB=Mean+3Standard Deviation). The limit-of-detection (LOD, defined as the lowest concentration of the target that can be reliably distinguished from the LOB and at which detection is feasible) was calculated using the linear regression equation of the calibration curve and the LOB.

Storage stability assessment: The storage stability of the e-Chip and the DNAzyme-conjugated mMB was investigated over an about 30-day storage period. The CP-deposited e-Chips were vacuum sealed and kept at about 4° C. over the course of this study. At time points of about 5, about 15 and about 30 days, e-Chips were removed from the vacuum and tested for the detection of an E. coli load of about 10⁵ CFU/mL using freshly synthesized DNAzyme-mMBs. The storage stability of the DNAzyme-mMBs was studied in two ways: (1) suspending about 10 mg/mL mMBs conjugated with about 10 μM DNAzyme (about 1 μL total volume) in modified buffer (about 4 μL: HEPES—about 1M, NaCl—about 150M, EDTA—about 5M, Tween 20—about 0.02%, about pH=7.2) and storing under vacuum sealed conditions. On about days 5, 15, and 30 of storage, the DNAzyme-mMB suspension was mixed with an E. coli load of about 10⁵ CFU/mL (about 5 μL total volume) and tested on freshly prepared e-Chips; or (2) lyophilizing about 1 mg/mL mMBs conjugated with about 1 μM DNAzyme and storing the dry product under vacuum sealed conditions. On about days 5, 15, and 30 of storage, the lyophilized pellet was resuspended in the DI water (about 5 μL) by hand shaking, mixed with an E. coli load of about 10⁵ CFU/mL (about 5 μL), and tested on freshly prepared e-Chips.

Statistical analysis: Data shown in the bar and scatter plots are presented as the mean±s.d., with sample sizes indicated for each relevant experiment. Comparisons between groups were made using a two-tailed Student's t-test. A p value of <0.05 was considered statistically significant.

### Results and Discussion

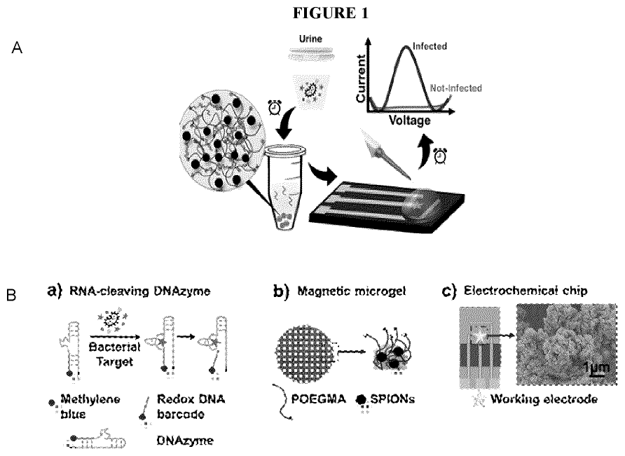

Developing the assay building blocks: A schematic showing an example of the assay process described herein from sample collection to assay result readout is shown in FIG. 1A. The bacterial detection device for use in the assay described herein—featuring distinct surfaces for target capture and signal transduction—was developed by integrating three functional building blocks (FIG. 1B): RNA cleaving DNAzymes (DNAzymes), microgel magnetic beads (mMBs), and a signal-transducing electrochemical chip (e-Chip).

DNAzymes are a class of functional nucleic acids and can be selected in vitro for specifically identifying bacterial targets, in this case protein targets released by Escherichia coli (E. coli); interaction between the DNAzyme and the bacterial proteins result in cleavage of the DNAzyme to release a DNA barcode that can be subsequently detected.^{1,2} The mMBs are designed as a colloidal support for presenting the DNAzyme probes for selective bacterial target capture. The e-chips are designed to capture the barcodes released from DNAzyme-functionalized mMBs and generate an electrochemical signal in response. The rationally designed device architecture allows for target capture and electrochemical signal transduction to occur on different surfaces; more specifically, the hydrogel-based interface used for target capture improving DNAzyme-target interactions and minimizing fouling can be decoupled from the electrode on which the presence of anti-fouling coatings can significantly reduce signal transduction and sensor sensitivity.

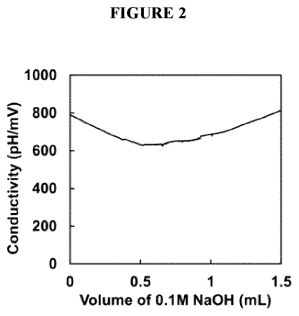

POEGMA mMBs were fabricated by copolymerizing oligo(ethylene glycol methacrylate) (n=7-8 ethylene oxide repeating units in the side chain), methacrylic acid (to provide functional groups enhance colloidal stability and facilitate ligand tethering through Schiff's base chemistry), and ethylene dimethacrylate (crosslinker) in the presence of pre-formed superparamagnetic iron oxide nanoparticles (SPIONs) via a semi-batch inverse suspension polymerization strategy. Conductometric base-into-acid titration (FIG. 2) indicated an experimental about 56 mol % methacrylic acid functionalization in the resulting mMBs, providing ample functional sites for ligand conjugation.

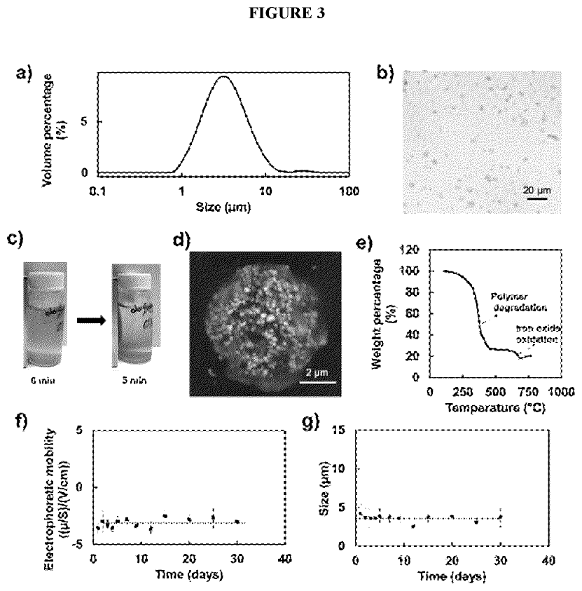

The fabricated mMBs exhibited a relatively narrow size distribution with an average diameter of about 5 μm (FIG. 3(a), 3(b)); this size was optimized to enhance surface area and colloidal stability while still facilitating rapid magnetic separation from a suspension using a relatively weak magnetic field (˜5 minutes with an inexpensive neodymium magnet, FIG. 3(c)). Scanning electron micrographs show a relatively uniform SPION distribution inside microgel (FIG. 3(d)), with thermogravimetric analysis indicating that ˜22 wt % of the mass of the dried microgel beads was attributable to SPIONs (FIG. 3(e)). Both the electrophoretic mobility (FIG. 3(f)) and the particle size (FIG. 3(g)) remained stable over about a one-month storage period, suggesting that the mMBs maintain high colloidal and degradative stability upon storage as is essential for the use of these mMBs in practical POC biosensors.

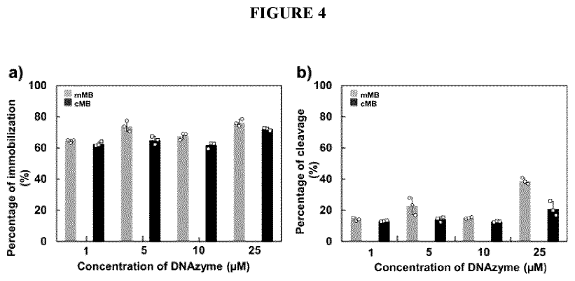

To functionalize the mMBs with biorecognition agents for developing a biosensor, the carboxylic acid groups from the methacrylic acid residues were grafted using carbodiimide chemistry with an amine terminated DNAzyme programmed to release a redox DNA barcode when it interacts with E. coli. A fluorescence test was used to verify and quantify DNAzyme biofunctionalization of the mMBs, indicating an estimated grafting of ˜5700 pmol DNAzymes/mg mMB bead. This graft density is on par with or (at higher DNAzyme concentrations) significantly higher than that achieved with commercial Dynabeads commonly used for DNA immobilization (FIG. 4).

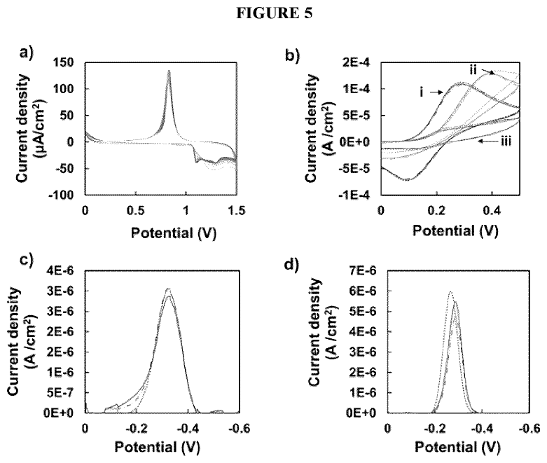

The e-Chip is a miniaturized three-electrode electrochemical cell with a hierarchically-structured working electrode designed to enhance the limit-of-detection of the electrochemical biosensor.³ The working electrode was functionalized with a capture probe and tested for reproducibility by performing three independent deposition steps (FIG. 5(a), 5(b)). The capture probe is designed to hybridize with the DNA barcode released in response to the bacterial target-DNAzyme interaction. By modifying the DNA barcode with a redox label (methylene blue), an electrochemical signal can be generated upon its capture on the working electrode.

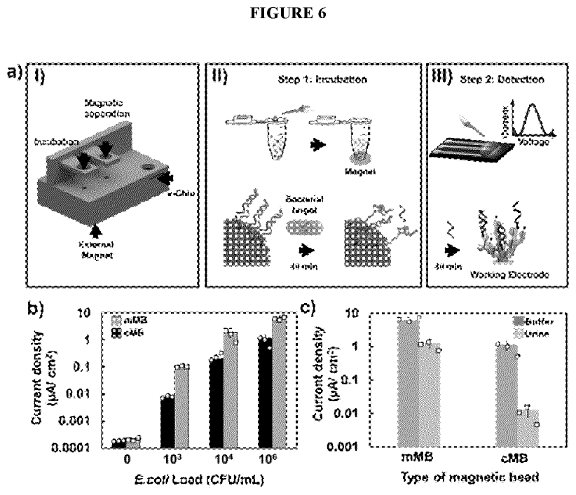

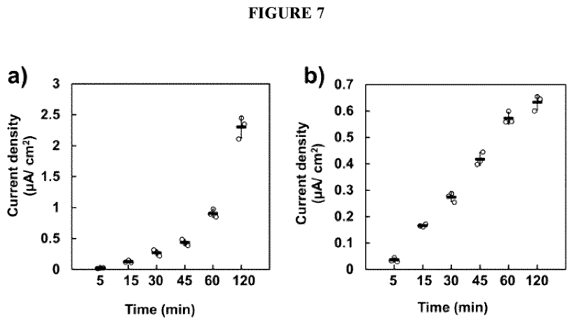

Validating the E. coli mMB kit: The mMBs were subsequently integrated into an electrochemical sensing platform based on a 3D-printed biosensing kit, referred to as the E. coli mMB kit. The E. coli mMB kit is comprised of an incubation slot, a magnetic separation slot, and the e-Chip slot (FIG. 6(a)-(I)). The assay was operated by adding the undiluted sample to the incubation tube containing the DNAzyme-grafted mMBs to allow for cleavage of the DNA barcode in the presence of bacteria (FIG. 6(a)-(II) step 1). Following this, the incubation tube was moved to the magnetic separation slot in which the mMBs were magnetically separated and the resulting supernatant was deposited on the e-Chip. In the presence of the target bacterium (in this case E. coli), the DNA barcodes present in the supernatant hybridize to the capture probes immobilized on the working electrode to generate a redox peak attributed to the reduction of methylene blue redox label present on the DNA barcode (FIG. 6(a)-(III) step 2). Highly consistent assay results were observed upon testing three independently fabricated e-Chips (FIG. 5(c)) and three independently fabricated DNAzyme-mMB conjugate batches (FIG. 5(d)), confirming the high batch-to-batch reproducibility of the assay. Kinetics studies of both the E. coli target interaction with DNAzyme (FIG. 7(a)) and the capture of released DNA barcode on the e-Chip (FIG. 7(b)) indicated optimum times of about 30 min. for both steps, resulting in a total about one-hour assay time amenable to point-of-care use.

The E. coli mMB kit was tested using both the mMBs developed in this work and commercially-available magnetic beads (Dynabeads, cMB) in the presence of different concentrations of E. coli in buffer. (FIG. 6(b)). A proportionate increase in the electrochemical signal was observed as the bacterial load was increased from about 10³ CFU/mL to about 10⁶ CFU/mL using either mMB or cMB; however, the electrochemical signal measured with mMBs was about 1000 times higher than that observed with cMBs at a bacterial load of about 10³ CFU/mL, with the blank signals being similar for both magnetic beads. The resulting about 1000-fold increase in signal-to-blank ratio obtained using mMBs as compared to cMBs motivated further exploration of the benefits of mMB for use in complex biological sample.

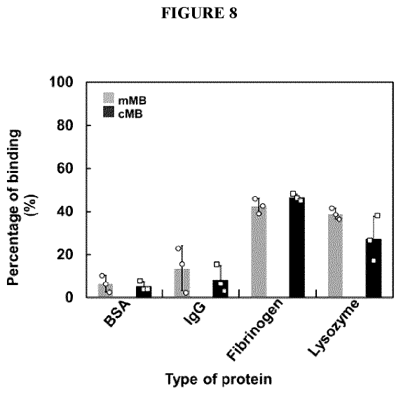

To assess the suitability of the mMBs in direct bacterial analysis in unprocessed biological samples, we compared the performance of mMBs and cMBs in unprocessed and undiluted human urine samples (FIG. 6(c)). Even though both cMBs and mMBs showed a reduced signal in urine relative to in buffer, the amount of signal reduction observed with mMBs (about 10× reduction) is significantly lower than that observed with cMBs (about 2000× reduction); indeed, the signal achieved with mMBs in undilute urine matched that of the cMBs in buffer (FIG. 6(c)). It is hypothesized that the combination of the inherent porosity (and thus the increased available surface area for DNAzyme conjugation), the enhanced hydration around the grafted DNAzyme to maintain it in its native conformation, the improved three-dimensional accessibility of the DNAzyme for bacteria interactions, and the reduced biofouling enabled by the POEGMA-based mMBs to be responsible for the increased signal-to-blank ratios achieved with mMBs compared to cMBs. The fluorescent protein adsorption experiments (FIG. 8) suggest that the improved performance may not be directly related to the improved protein repellency of mMBs, with the uptake of lysozyme, bovine serum albumin (BSA), immunoglobin G (IgG), and fibrinogen (chosen based on the prevalence of these proteins in biological samples and their broad range of shapes, molecular weights, and surface charges) observed to be similar between mMBs and cMBs.

Without being bound by theory, unlike the “hard” cMBs, mMBs can both adsorb protein on their surface as well as absorb protein into the hydrated gel network, with the protein binding assay being unable to discriminate between the two; if a significant portion of the protein uptake into mMBs occurs via absorption rather than adsorption, the proteins present are much less likely to inhibit DNAzyme-target interactions. However, overall, the data suggest that the significantly improved surface hydration of the mMBs relative to the cMBs (and the resulting easier access of the E. coli target to the DNAzyme binding site) is the primary mechanism by which the large improvement in assay sensitivity in urine is achieved.

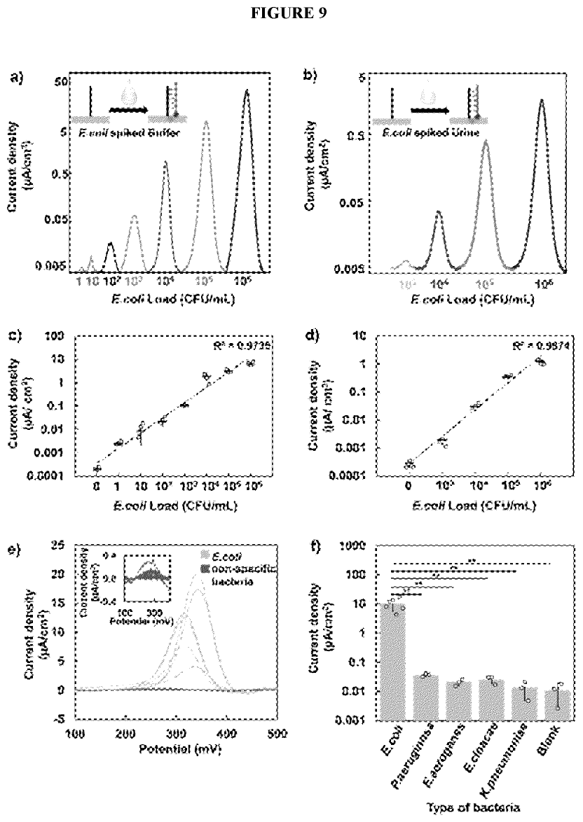

E. coli mMB kit for detecting urinary tract infections: Urinary tract infections (UTIs) are the most common infections treated in primary health settings making their rapid, point-of-care, and effective diagnosis critically important. E. coli is the leading microorganism causing UTIs, and its detection and identification in urine has important clinical diagnostic and ultimate therapeutic value. To evaluate the analytical performance of the E. coli MB kit, buffer and unprocessed human urine samples were spiked with different concentrations of E. coli targets obtained from laboratory growth cultures. Electrochemical measurements (FIGS. 9(a), 9(b)) indicate that the E. coli mMB kit is highly sensitive to the presence, absence, and concentration of the E. coli present in the solution. Further analysis of the data using a calibration plot indicates a limit-of-detection of about 6 CFU/mL (sensitivity: about 1.9 μA/cm²·log (CFU/mL)) in buffer and about 138 CFU/mL (sensitivity: about 0.4 μA/cm²·log (CFU/mL)) in urine (FIGS. 9(c), 9(d)). As such, the E. coli mMB kit accurately detects E. coli in urine at concentrations directly relevant for early detection of UTIs in symptomatic patients (≥10³ CFU/mL) with a significantly shorter sample-to-result time (˜1 hour) compared to the currently-used methods based on urine growth cultures (about 16 to about 48 hr). It should be noted that even though antibiotics are typically prescribed for UTIs when growth cultures demonstrate E. coli concentrations of >10⁵ CFU/mL, bacteria have been detected in ˜90% of the “no growth” (<10⁵ CFU/mL) urine cultures for symptomatic patients; as such, the very low limit-of-detection of the E. coli mMB kit is highly desirable for UTI management.

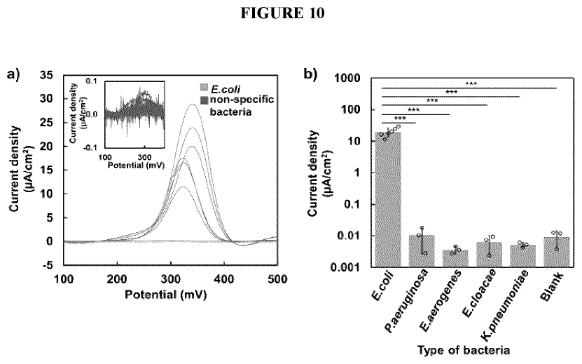

A panel of urinary bacterial pathogens was chosen to validate the specificity of the E. coli mMB kit to determine the potential utility of the kit for bacterial identification. Klebsiella pneumoniae, Enterobacter aerogenes, Enterobacter cloacae, and Pseudomonas aeruginosa were selected as potential interfering bacteria due to their roles as other common sources of UTIs that need to be treated with alternative antibiotic regimens. To verify specificity, a high load of bacteria from growth cultures (about 10⁶ CFU/mL) was spiked into both buffer and unprocessed human urine. A strong electrochemical signal was observed when the sensor was exposed to its intended E. coli target spiked in buffer; in contrast, non-targeted bacteria showed no difference in the signal relative to the measurements obtained from the sample with no bacteria (blank sample) (FIG. 10(a), 10(b)). Similarly, in spiked urine, E. coli outputted a signal that was >2 orders of magnitude higher than the blank while all the other bacteria tested did not show any significant signal relative to the blank (FIG. 9(e), 9(f)). These results demonstrate the high specificity of the E. coli mMB kit and thus its ability to specifically identify the targeted bacterium.

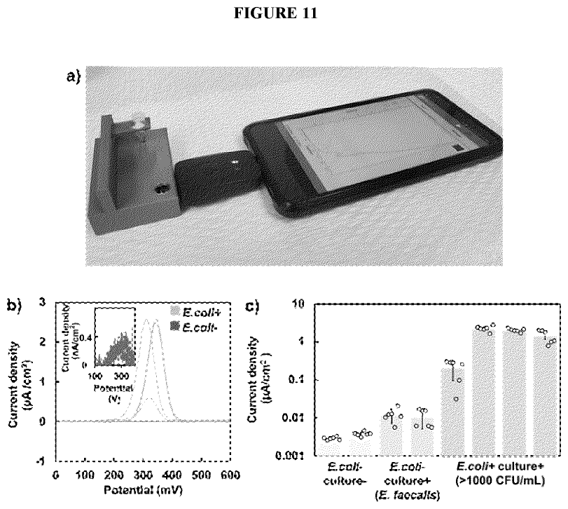

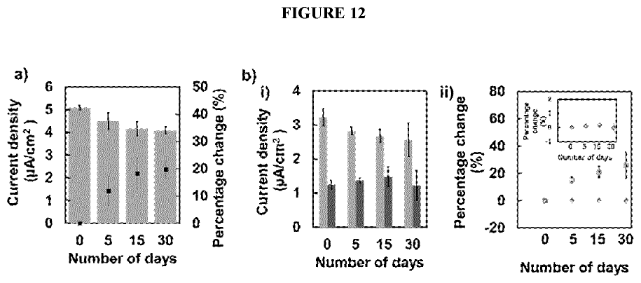

Clinical performance of the E. coli mMB kit: To directly evaluate the potential clinical applicability of the E. coli mMB kit, three types of patient-acquired urine samples (as classified by conventional urine growth cultures) were analyzed: (1) E. coli+/culture+ (infected with a clinically-significant level, >1000 CFU/mL, of E. coli); (2) E. coli−/culture+ (infected with clinically-significant level of E. faecalis but no significant amount of E. coli); and (3) E. coli−/culture− (no clinically-significant level of any bacteria). The urines being analyzed were added to the reaction tubes and, following magnetic separation, were dropped on the e-Chips for signal transduction and readout (FIG. 11(a)). The E. coli+/culture+ samples generated an electrochemical current >0.2 μA/cm² (FIG. 11(b), 11(c)); based on the calibration curves in FIG. 9(d), this signal corresponds to the E. coli load of about 10⁴ to about 10⁵ CFU/mL that is directly consistent with readings obtained from the growth cultures. In comparison, the signals observed from E. coli−/culture+ and E. coli−/culture− samples were ˜20 fold lower (≤0.01 μA/cm²), consistent with the signals observed for the blank and negative urine signal levels in the analytical (FIG. 9(b)-9(d), blank signal <0.001 μA/cm²) and specificity (FIG. 9(e), 9(f), blank signal <0.01 μA/cm², negative control <0.01 μA/cm²) experiments. As such, the E. coli mMB kit can successfully classify unprocessed clinical urine samples as E. coli+ or E. coli− and accurately quantify the bacterial load in the sample using two easy (add sample+measure) steps without any need for sample processing or the addition of reagents. The storage stability of both the e-Chip and the DNAzyme-mMBs was tested over about a 30-day period to assess the practical translatability of the assay. Vacuum-packed e-Chips stored in the fridge demonstrated about a 20% decrease in the signal after about 30 days of storage (FIG. 12(a)), in agreement with reported literature on storage stability of similarly functionalized gold electrodes.⁴ The storage stability of the DNAzyme-mMB was tested by storing the beads in suspension in a DNAzyme-stabilizing buffer or lyophilizing and then redispersing them in RNAse-free water from the dry state when required. Suspension storage results in a ˜25% signal reduction, attributable to trace DNAse/RNAse in the suspension buffer; in contrast, while the maximum signal in the lyophilized DNAzyme-mMBs is lower upon redispersion, the signal exhibits no change whatsoever over the about 30-day storage period. Given the very high signal to background ratio of this assay, any combination of these storage activity losses would still comfortably enable the correct classification of infected versus uninfected urine at the clinical threshold (about 1000 CFU/mL), suggesting that the assay has potential translatability.

### CONCLUSION

Since the invention of the glucose monitor, electrochemical biosensors have gained attention due to their ability to perform ultrasensitive signal readout using simple, cost-effective, and portable instruments. A major challenge in the use of electrochemical readout in bioanalysis is the requirement for sample processing, stringent washes, and analyte amplification for mitigating or compensating for the signal loss (relative to the background signal) that occurs in native clinical and biological samples and compromises the sensitivity of the biosensor. In this disclosure, in embodiments, the biorecognition step can be separated from the signal transduction step (which are traditionally performed on a single surface) into distinct surfaces designed to enhance signal:noise in each step of the assay: for example, biorecognition on the surface of highly hydrated and porous microgel magnetic beads (mMBs) and signal transduction on the hierarchical surface of a working electrode on an e-Chip. Programmable self-cleaving DNAzymes immobilized on the mMBs can enable the connection of biorecognition on mMBs with signal transduction on e-Chips through the release of a DNA barcode. The anti-fouling and hydrated porous interface provided by mMBs can enable the bacteria to interact more effectively with DNAzymes compared to commercially-available magnetic beads, which can increase the amount of the DNA barcode that was released and ultimately measured on the e-Chip. This improvement can ultimately enhance the signal-to-background ratio and thus lower the limit-of-detection of the electrochemical assay by reducing the signal loss typically observed in analyzing clinical samples. This can enable the reagent addition-free, wash-free, and amplification-free detection of specific bacteria in a complex biological fluid. While multiple types of “low-fouling” commercial magnetic beads are available, no commercial magnetic bead is based on a hydrogel for this application. This lack of commercial microgel-based beads is likely attributable to the synthetic challenges inherent in creating monodisperse, colloidally stable, and well-defined microgel particle populations on the about 1 to about 10 μm size range that have an ideal balance of high surface area, colloidal stability (challenging to maintain in magnetic microgels that can be due to the high-density and self-aggregation prone SPIONs), and rapid magnetic separation potential. The semi-batch inverse emulsion templating strategy described herein can significantly suppress aggregation while also achieving smaller particle sizes relative to previously reported microgel bead fabrication strategies.⁵ The facile functionalization of the microgel magnetic beads via copolymerization may also enable facile control over the loading density of DNAzyme on the microgel magnetic beads for achieving optimal biosensing performance.

In spite of a loss in signal in clinical samples compared to buffer solutions, the high inherent signal-to-background ratio of the E. coli mMB kit (enabled by the specific properties of the mMBs) still allowed, for example, a limit-of-detection (about 138 CFU/mL) in undiluted urine without any sample processing, target amplification, or manual washes, enabling the clinical diagnosis of UTI; furthermore, the high selectivity of DNAzyme-based sensors for their target coupled with the hydrated microenvironment provided by the mMBs can allow for specifically distinguishing E. coli-containing urines from human urine samples containing other urinary bacteria. Such benefits can allow for the point-of-care implementation of the E. coli mMB kit for rapid on-site bacterial identification and thus selection of the most appropriate therapeutic intervention. The geometry of the E. coli mMB kit can also be amenable to multiplexing, as multiple DNAzymes are now available for various pathogens, and they can be designed, programmed, and integrated into the mMBs to release specific DNA barcodes. Such a strategy could be extended to the rapid identification of a panel of pathogens for managing both UTIs as well as a wide range of other infectious diseases.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### TABLES

TABLE 1
Summary of the oligonucleotides used for DNAzyme-based E. coli detection.

| SEQ ID NO. | Name | Sequence (5′-3′) |
|---|---|---|
| 1 | MB-DNAzyme | MB-TTTTTTGTGTGACTCTTCCTAGCTrATGGTTC |
| | (79 nt) | GATCAAGAGATGTGCGTCTTGATCGAGACCT |
| | | GCGACCGTTTTTTTTTT-amine |
| 2 | FAM-DNAzyme | TTTTTTGTGTGACTCTTCCTAGCTFAMrATGGTT |
| | (79 nt) | CGATCAAGA GATGTGCGTCTTGATCGAGA |
| | | CCTGCGACCGTTTTT TTTTT-amine |
| 3 | Capture probe (CP) | TAGCTAGGAAGAGTCACACA-SH |
| | (20 nt) | |
| 4 | Redox probe (R) | MB-TTTTTTGTGTGACTCTTCCTAGCTrA |
| | (25 nt) | |

MB = 5′-Methylene blue; amine = 3′-Amine; rA = riboA; FAM = 6-fluorescein; SH = 3′-Thiol

### CITATIONS

(1) Ali, M. M., Aguirre, S. D., Lazim, H., & Li, Y. (2011). Fluorogenic DNAzyme Probes as Bacterial Indicators. Angewandte Chemie—International Edition, 50(16), 3751-3754.
(2) Pandey, R.; Chang, D.; Smieja, M.; Hoare, T.; Li, Y.; Soleymani, L. (2021). Integrating Programmable DNAzymes with Electrical Readout for Rapid and Culture-Free Bacterial Detection Using a Handheld Platform. Nat. Chem. 13, 895-901.
(3) Traynor, S. M.; Wang, G. A.; Pandey, R.; Li, F.; Soleymani, L. (2020). Dynamic Bio-Barcode Assay Enables Electrochemical Detection of a Cancer Biomarker in Undiluted Human Plasma: A Sample-In-Answer-Out Approach. Angew. Chemie—Int. Ed. 130(50), 22806-22811.
(4) Kuralay, F.; Campuzano, S.; Wang, J. (2012). Greatly Extended Storage Stability of Electrochemical DNA Biosensors Using Ternary Thiolated Self-Assembled Monolayers. Talanta 99, 155-160.
(5) Hernández-Barajas, J.; Hunkeler, D. J. (1997). Inverse-Emulsion Copolymerization of Acrylamide and Quaternary Ammonium Cationic Monomers with Block Copolymeric Surfactants: Copolymer Composition Control Using Batch and Semi-Batch Techniques. Polymer (Guildf). 38(2), 449-458.

1.-52. (canceled)

53. A magnetic microparticle comprising a magnetic nanoparticle encapsulated by a polymer hydrogel.

54. The magnetic microparticle of claim 1, wherein the polymer hydrogel comprises a three-dimensional crosslinked network of water-soluble polymer(s).

55. The magnetic microparticle of claim 1, wherein one or more of the following:
the polymer hydrogel comprises a protein repellent polymer;
the polymer hydrogel comprises poly(oligo(ethylene glycol) methacrylate or a poly(ethylene glycol) derivative;
the polymer hydrogel comprises a zwitterionic polymer; optionally, the zwitterionic polymer is selected from the group consisting of polysulfobetaine(s), poly(sulfobetaine) methacrylate, polycarboxybetaine(s), poly(carboxybetaine) methacrylate, and poly(phosporylcholine); and
the polymer hydrogel comprises poly(N-vinylpyrrolidone), poly(acrylamide) and poly(acrylamide) derivatives, polyglycidol and polyglycidol derivatives, or poly(2-oxazoline) or poly(2-oxazoline) derivatives.

56. The magnetic microparticle of claim 1, wherein the microparticle is a microgel.

57. The magnetic microparticle of claim 4, wherein the microgel comprises at least one dimension on the length scale of about 10 nm to about 1000 μm, optionally, at least about 5 μm.

58. The magnetic microparticle of claim 1, wherein the magnetic nanoparticle comprises iron oxide.

59. The magnetic microparticle of claim 1, wherein the microparticle is from about 0.5 μm to about 100 μm in diameter, optionally, at least about 5 μm in diameter.

60. The magnetic microparticle of claim 1, wherein the microparticle is prepared by inverse emulsion templating.

61. The magnetic microparticle of claim 1, further comprising a biorecognition agent functionalized on and/or in the microparticle.

62. The magnetic microparticle of claim 9, wherein the biorecognition agent is at least one of a DNAzyme, an aptamer, and an antibody.

63. An assay for detecting the presence of a target analyte in a sample comprising:
a) the magnetic microparticle of claim 9, wherein the biorecognition agent further comprises a reporter moiety;
b) an electrochemical chip comprising a working electrode, a counter electrode and a reference electrode; and
c) a capture probe functionalized on the working electrode;
wherein binding of the biorecognition agent to the target analyte results in production of an electrochemical, electroluminescent or photoelectrochemical signal.

64. The assay of claim 11, wherein one or more of the following:
the working electrode comprises a conductive material, semi-conductive material, or a combination thereof;
the working electrode comprises metal;
the working electrode comprises gold; or
the working electrode further comprises hierarchical structures.

65. The assay of claim 11, wherein one or more of the following:
the electrochemical signal is measured by amperometry, voltammetry, photoelectrochemistry, electrochemiluminescence, potentiometry or impedance;
the biorecognition agent is at least one of a DNAzyme, an aptamer, and an antibody;
the reporter moiety comprises at least one of a redox species, a photoactive species, and a electrochemiluminescence species, optionally, wherein the redox species is methylene blue;
the reporter moiety comprises a biopolymer modified with the redox species, optionally, wherein the biopolymer comprises single-stranded DNA;
the capture probe comprises single-stranded DNA;
the target analyte comprises a microorganism target, optionally, wherein the microorganism is Escherichia coli;
the sample is a urine sample, optionally, an unprocessed urine sample;
the target analyte is detected in the sample in an amount of about 10 CFU/mL to about 106 CFU/mL;
the assay has a limits-of-detection for the target analyte of about 50 CFU/mL to about 200 CFU/mL, optionally, of about 138 CFU/mL;
the assay is performed within about 30 minutes to about 10 hours; about 30 minutes to about 8 hours; about 30 minutes to about 7 hours; about 30 minutes to about 6 hours; about 30 minutes to about 5 hours; about 30 minutes to about 4 hours; about 30 minutes to about 3 hours; about 30 minutes to about 2 hours; about 30 minutes to about 1 hour; about 45 minutes to about 1 hour; or about 1 hour;
the assay is for use in screening and/or diagnostics, treatment monitoring, environmental monitoring, health monitoring, and/or pharmaceutical development; and
the assay detects a urinary tract infection in a subject.

66. A kit for detecting the presence of a target analyte in a sample, wherein the kit comprises
a) the magnetic microparticle of claim 9, wherein the biorecognition agent further comprises a reporter moiety;
b) an electrochemical chip comprising a working electrode, a counter electrode and a reference electrode;
c) a capture probe functionalized on the working electrode;
d) a magnet; and
e) instructions for use of the kit.

67. The kit of claim 14, further comprising a sample container and an electrical reader.

68. A method of determining the presence of a target analyte in a sample comprising:
a) exposing the magnetic microparticle of the assay of claim 11 to the sample to release the reporter moiety from the biorecognition agent in the presence of the target analyte;
b) separating the magnetic microparticle from the sample; and
c) depositing the sample of step b) to the electrochemical chip of the assay of claim 11;
wherein the capture probe of the assay of claim 11 binds the reporter moiety to produce an electrochemical signal.

69. The method of claim 16, wherein one or more of the following:
the electrochemical signal is measured by square wave voltammetry;
the magnetic microparticle is exposed to the sample under conditions for binding the biorecognition agent to the target analyte;
the biorecognition agent is at least one of a DNAzyme, an aptamer, and an antibody;
the target analyte comprises a microorganism target, optionally, wherein the microorganism is Escherichia coli;
the sample is a urine sample; or
the method detects a urinary tract infection in a subject.

70. The magnetic microparticle of claim 1 for capturing a target analyte in a sample.

71. The magnetic microparticle of claim 1 for determining the presence of a target analyte in a sample.

72. The assay of claim 11 for determining the presence of a target analyte in a sample.


SEQUENCE LISTING (4 sequences)

SEQ ID NO: 1 - DNA, 79 bp, synthetic construct
  source 1..79: mol_type = other DNA
  misc_feature 1..24: note = DNA
  misc_feature 25: note = RNA
  misc_feature 26..79: note = DNA
  misc_binding 1: bound_moiety = methylene blue
  misc_binding 79: bound_moiety = amine
ttttttgtgtgactcttcctagctatggttcgatcaagagatgtgcgtcttgatcgagacctgcgaccgtttttttttt

SEQ ID NO: 2 - DNA, 79 bp, synthetic construct; identical to SEQ ID NO: 1
  source 1..79: mol_type = other DNA
  misc_feature 1..24: note = DNA
  misc_feature 25: note = RNA
  misc_feature 26..79: note = DNA
  misc_binding 24: bound_moiety = 6-fluorescein
  misc_binding 79: bound_moiety = amine

SEQ ID NO: 3 - DNA, 20 bp, synthetic construct
  source 1..20: mol_type = other DNA
  misc_binding 20: bound_moiety = thiol
tagctaggaagagtcacaca

SEQ ID NO: 4 - DNA, 25 bp, synthetic construct
  source 1..25: mol_type = other DNA
  misc_feature 1..24: note = DNA
  misc_feature 25: note = RNA
  misc_binding 1: bound_moiety = methylene blue
ttttttgtgtgactcttcctagcta